(19) United States
(12) Patent Application Publication
(10) Pub. No.: US 2025/0306043 A1
(43) Pub. Date: Oct. 2, 2025

(54) FECAL SAMPLING AND ANALYSIS SYSTEM

(71) Applicant: Alexey Melnik, Tolland, CT (US)

(72) Inventor: Alexey Melnik, Tolland, CT (US)

(21) Appl. No.: 19/092,773

(22) Filed: Mar. 27, 2025

Related U.S. Application Data
(60) Provisional application No. 63/570,322, filed on Mar. 27, 2024.

(51) Int. Cl. G01N 33/92; G01N 1/34

(52) U.S. Cl. CPC G01N 33/92; G01N 1/34

(57) ABSTRACT

A vial adapted for preservation of a metabolite profile of a fecal sample during transport thereof is provided. The vial includes a body including a volume of preservative; and, a cap configured to engage with the body and to seal sample fluid including a mixture of the preservative and fecal sample within the vial during transport by a common carrier. A kit and a system are included.

### CROSS REFERENCE TO RELATED APPLICATIONS

The invention disclosed herein claims priority to U.S. Provisional Patent Application No. 63/570,322, entitled “Fecal Sampling and Analysis System,” filed on Mar. 27, 2024, the entire disclosure of which is incorporated herein in it's entirety.

### BACKGROUND OF THE INVENTION

### 1. Field of the Invention

The invention disclosed herein relates to methods and apparatus for performing fecal sampling, and in particular to techniques which rapidly translate to the analysis environment.

### 2. Description of the Related Art

It is easy to take the benefit of Western medicine for granted. However, many populations do not have access to the tools available in the West. For example, various sampling and analysis protocols require substantial infrastructure. Consider techniques that require constant refrigeration post sampling up to points of analysis. In many impoverished regions, the requisite “cold chain” is simply not available or reliable enough to yield meaningful data, and therefore use of the sampling techniques is not feasible. It is known that a great deal of information about the health of an individual may be obtained by analysis of fecal samples, yet sampling techniques that would provide research and clinical insights are not available with standard methods for such problematic populations.

Fecal collection techniques that are inexpensive and easy to implement can provide greater access to tools for monitoring health conditions. Robust systems for fecal sampling can result in more routine and personalized insight into gut health, leading to better monitoring and management of health conditions.

Consider, for example, that improved techniques can enable users and patients to more frequently undertake personalized sampling. This may lead to improved monitoring of related data such as needed to obtain or characterize dynamics of the microbiome in the gut of the patient. That is, frequent snapshots of an individual's microbiome could help optimize therapies and gut health interventions. Increasingly, it has been recognized that the microbiome in the gut of an individual plays important roles in cognition and immunological responses.

As a research tool, low-burden, inexpensive techniques could facilitate large-scale epidemiological studies exploring links between lifestyle, diet, gut microbiome, and health outcomes across populations. Such techniques would allow fecal sampling from populations where traditional techniques are challenging, such as pediatric patients who often fail to provide samples.

Further, improved techniques may improve sampling with difficult patients. For example, for patients with autism spectrum disorder, gastrointestinal disturbances are common but difficult to monitor due to compliance challenges with collection protocols. More accessible methods could improve diagnosis and treatment. It is well-known that many patients are simply non-compliant with medical instructions when a level of difficulty, discipline or complexity is considered to be too demanding.

Further, it is recognized that many sampling techniques simply result in qualitative data. That is, non-standardized or inaccurate sampling protocols result in inaccurate test results. For example, when a sample size is not well-characterized, it is difficult or impossible to ascertain concentrations of targeted molecules or biota.

Thus, what are needed are easy, fast, low-cost approaches for fecal sample collection. Preferably, the techniques provide for accurate analysis results among underserved communities lacking access to traditional clinical tests.

### SUMMARY OF THE INVENTION

In one embodiment, a vial adapted for preservation of a metabolite profile of a fecal sample during transport thereof is provided. The vial includes a body including a volume of preservative; and, a cap configured to engage with the body and to seal sample fluid including a mixture of the preservative and fecal sample within the vial during transport by a common carrier.

The vial may be configured for receiving a swipe adapted for retention of the fecal sample. The metabolite profile may be associated with at least one of a type of pathogen, a type of bacteria, a type of virus, a gastrointestinal infection, an inflammatory bowel disease, a parasite, a digestive disorder, a gut microbiome, and cancer. The preservative may include at least one of water, ethanol, methanol, sodium acetate, formalin, polyvinyl alcohol (PVA), a Cary-Blair medium and sodium thioglycolate. At least one of the body and the cap may include at least one of dimples, detents, ridges, splines, threads, a snap, an interlock and at least one sealing feature, and the sealing feature may include at least one a sealing ring and a separate washer. The body may include a cover adapted for retention of the preservative until installation of the cap, thereupon causing a free-flow of the preservative between the cap and the body. The cap may include an engagement feature for causing breakage of the cover. A plunger may be included and configured for compressing a swipe containing the fecal sample upon centrifugation of the vial. The body may be configured for receiving a swipe containing the fecal sample. The cap may include a chamber configured for receiving a swipe containing the fecal sample. The body may be configured for evaluation by analytical equipment of a laboratory.

In another embodiment, a kit for collecting a fecal sample is provided. The kit including a vial adapted for preservation of a metabolite profile of a fecal sample during transport thereof, the vial including: a body including a volume of preservative; and, a cap configured to engage with the body and to seal sample fluid including a mixture of the preservative and fecal sample within the vial during transport by a common carrier; and, a swipe for collection of the fecal sample, the swipe configured for being loaded into the vial. The kit may further include at least one of hygienic accessories and a shipping container. The swipe may be substantially lint-free. The kit may further include a data set.

In another embodiment, a system for ascertaining metabolite profile information from a fecal sample is provided. The system includes sample handling equipment for analytical preparation of the fecal sample, the fecal sample provided in a vial configured for transport by a common carrier, the vial including a body and a cap engaged with the body and sealing sample fluid including a mixture of preservative and a swipe with the fecal sample therein; analysis equipment suited for analyzing the prepared fecal sample; and a processor including machine executable instructions stored on non-transitory machine readable media, the instructions for at least one of operating the sample handling equipment, operating the analysis equipment, interpreting output of the analysis equipment and providing a report containing the metabolite profile information.

The analysis equipment may be configured for at least one of gas chromatography-mass spectrometry (GC-MS); liquid chromatography-mass spectrometry (LC-MS); nuclear magnetic resonance (NMR) spectroscopy; and fourier transform infrared (FTIR) spectroscopy. The instructions may be configured to adjust the metabolite profile information according to a data set including information about at least one of the swipe and the sample fluid. The instructions may perform the interpreting by implementation of artificial intelligence (AI). The report may contain metabolite profile information containing at least one of molecular identity information, concentration information, and pathology information.

### BRIEF DESCRIPTION OF THE DRAWINGS

The features and advantages of the invention are apparent from the following description taken in conjunction with the accompanying drawings in which:

FIG. 1 is an abstraction depicting aspects of a patient-oriented process for sampling;

FIG. 2 depicts aspects of a first embodiment of a sampling device according to the teachings herein;

FIGS. 3A, 3B, 3C and 3D, collectively referred to herein as FIG. 3, depict aspects of another embodiment of a sampling device according to the teachings herein;

FIGS. 4A, 4B, and 4C, collectively referred to herein as FIG. 4, depict aspects of yet another embodiment of a sampling device according to the teachings herein;

FIG. 5 depicts aspects of a further embodiment of a sampling device according to the teachings herein;

FIG. 6 depicts aspects of a kit including a sampling device according to the teachings herein;

FIG. 7 depicts aspects of an exemplary system for analysis of samples as described herein;

FIGS. 8A through 8D, collectively referred to herein as FIG. 8, is a graphic depicting variability of different molecules across participants;

FIGS. 9A through 9D, collectively referred to herein as FIG. 9, are graphics related to comparative sampling techniques;

FIGS. 10A through 10C, collectively referred to herein as FIG. 10, are graphics correlating sample results for conventional techniques with those described herein; and,

FIG. 11 is a graphic depicting aspects of comparative data for shipping trials.

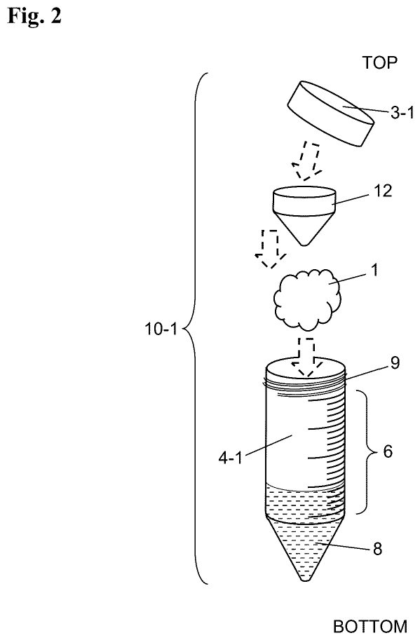

It should be noted that terms of orientation such as “top” and “bottom” as used in the drawings (see FIG. 2) are merely for ease of referencing and not to be construed as limiting of the teachings herein.

### DETAILED DESCRIPTION OF THE INVENTION

Disclosed herein are methods and apparatus for stool sample collection and preservation. Advantageously, the methods and apparatus provide for seamless translation into analysis protocols.

Generally, disclosed herein are methods and apparatus for low-cost at-home stool sample collection and preservation for laboratory analyses, such as analyses using mass spectroscopy systems. The techniques make use of substantially lint-free paper wipes and containers loaded with a preservative. Generally, the containers may be coupled with a sample storage device that provides for easy translation from sampling to sample storage. The combination of the container and sample storage device enables direct sampling and stabilization of stool specimens, which can then be mailed to a laboratory for analyses such as metabolomics profiling.

Use of the methods and apparatus disclosed herein enable easy, direct stool sampling without requiring bulky containers or stool collection devices. The techniques are low cost and provide accessibility for regular consumer health monitoring. The technology disclosed herein is a simplified at-home collection compared to traditional scoop methods and allows samples to be immediately stabilized after collection by direct immersion in preservative. The substantially lint-free wipes optimize sample recovery and avoid sample contamination. Preserved samples remain stable allowing room temperature shipping, thus avoiding complications of cold-chain transfer and storage. This method of collection is compatible with high-throughput automated sample processing workflows like metabolomics analysis providing functional biomarker insights into the microbiome.

Advantageously, by avoiding the complications of cold-chain transfer and storage, shipment of fecal samples may be accomplished through use of a “common carrier,” (that is, “standard” shipping protocols that move a majority of goods transported in commerce). Examples of common carriers include, without limitation, United Parcel Service (UPS), Federal Express (FedEx), the United States Postal Service (USPS), various private carriers or logistics companies and others. In short, use of the term “common carrier” is to be construed as a shipping technique that does not require the protections of a cold-chain.

Before discussing the technology in greater detail some concepts are introduced.

Generally, a “stool sample,” which may also referred to as a “fecal sample,” is a specimen of feces collected for diagnostic or investigative purposes. The stool sample includes a portion of excrement, typically expelled during a bowel movement, and which is collected in a sterile container for analysis. Stool samples serve as valuable biological material for assessing gastrointestinal health, identifying pathogens, monitoring digestive disorders, and screening for various medical conditions.

For example, a stool sample collected from a patient experiencing gastrointestinal discomfort may be analyzed to diagnose presence of Clostridium difficile, indicating a possible infection. A stool sample obtained from a child with persistent diarrhea may be tested for identification of possible for rotavirus. Analysis of a stool sample from an individual with suspected inflammatory bowel disease may be used to detect elevated levels of calprotectin. A stool sample may be analyzed to detect agents associated with a variety of forms of cancer.

In short, collection of a stool sample may be used for a variety of diagnostic purposes. Stool samples may be collected to diagnose gastrointestinal infections, inflammatory bowel diseases, parasites, and other digestive disorders. Stool samples may be collected to screen for pathogens. For example, healthcare providers may obtain stool samples to screen for bacterial, viral, and parasitic pathogens, such as Salmonella, norovirus, and Giardia. Stool samples may be collected to monitor digestive health. For example, stool analysis aids in monitoring the effectiveness of treatment for gastrointestinal conditions and assessing overall digestive health. Stool samples may be collected to support research and epidemiological studies. Researchers use stool samples to study the gut microbiome, investigate disease patterns, and conduct epidemiological research on gastrointestinal illnesses.

A variety of current collection take techniques are known. Of particular focus are home collection techniques. With the advent of telemedicine and remote healthcare services, patients may collect stool samples at home using provided kits and mail them to laboratories for analysis. Such kits may use any one or more of a number of commonly used preservatives.

Examples of preservatives include: ethanol, methanol, sodium acetate (to preserve stool samples for microbiological testing by inhibiting bacterial growth), formalin (which may be used for preserving stool samples for parasitological examinations, particularly for detecting intestinal parasites), polyvinyl alcohol (PVA) (which may be relied upon to maintain the morphology of parasites in stool samples for microscopic examination), Cary-Blair Medium (which preserves stool samples for culturing enteric pathogens such as Salmonella and Shigella) and sodium thioglycolate (which helps maintain anaerobic conditions in stool samples during transportation and storage, preserving anaerobic bacteria for analysis). Other preservatives or combinations thereof may be used. Other materials, such as water, may be included in the preservative.

As may be surmised, competitive preservatives exist for a reason. That is, one preservative may be better than another at preserving a particular quantity of interest within the stool sample. Generally, in some of the embodiments disclosed herein, ethanol is used as the preservative. It has been found that ethanol is particularly effective for preserving samples used in metabolomics analyses.

Generally, as discussed herein, “metabolomics” encompasses the study of metabolite profiles in biological samples, including tissues, blood, urine, and increasingly, stool samples. It employs advanced analytical techniques such as mass spectrometry (MS) and nuclear magnetic resonance (NMR) spectroscopy to detect, identify, and quantify metabolites. By profiling metabolites, metabolomics offers a holistic view of metabolic pathways, metabolic dysregulation, and metabolic phenotypes, enabling researchers to understand the biochemical basis of health and disease. Generally, as used herein, the term “metabolite profile” refers to a distribution of a variety of chemical and/or biological products associated with a particular fecal sample.

Stool samples have emerged as valuable surrogate media for metabolomic analysis due to their accessibility, non-invasive collection, and rich microbial and metabolic content. The human gut microbiota plays a crucial role in metabolizing dietary components, producing bioactive metabolites, and influencing host metabolism. Analyzing metabolites in stool samples allows researchers to investigate the dynamic interplay between the gut microbiome and host metabolism, shedding light on the role of the gut microbiota in health and disease.

In metabolomic analysis of stool samples, researchers can discover a wide range of metabolites representing various classes and biochemical pathways. Some of the quantities that may be discovered in sample analysis include: Short-Chain Fatty Acids (SCFAs): Metabolites produced by microbial fermentation of dietary fiber in the colon, including acetate, propionate, and butyrate, which play roles in energy metabolism, gut health, and immune regulation; Amino Acids: Building blocks of proteins and precursors for various metabolic pathways, amino acids such as tryptophan, phenylalanine, and tyrosine are detected in stool samples and can reflect dietary intake, protein metabolism, and microbial metabolism; Bile Acids: Metabolites derived from cholesterol and modified by gut microbiota and host enzymes, bile acids play essential roles in lipid digestion, cholesterol metabolism, and signaling pathways in the gut and beyond; Polyphenols and Phytochemicals: Bioactive compounds derived from plant foods, including flavonoids, phenolic acids, and lignans, which exhibit antioxidant, anti-inflammatory, and potential anticancer properties; Secondary Metabolites: Metabolites produced by microbial metabolism, including secondary bile acids, indole derivatives, and trimethylamine-N-oxide (TMAO), which can influence host metabolism, inflammation, and disease risk; and, Gut Microbial Metabolites: Metabolites produced by the gut microbiota, such as trimethylamine (TMA), short-chain fatty acids, and secondary bile acids, which modulate host physiology, immune function, and metabolic health (among others).

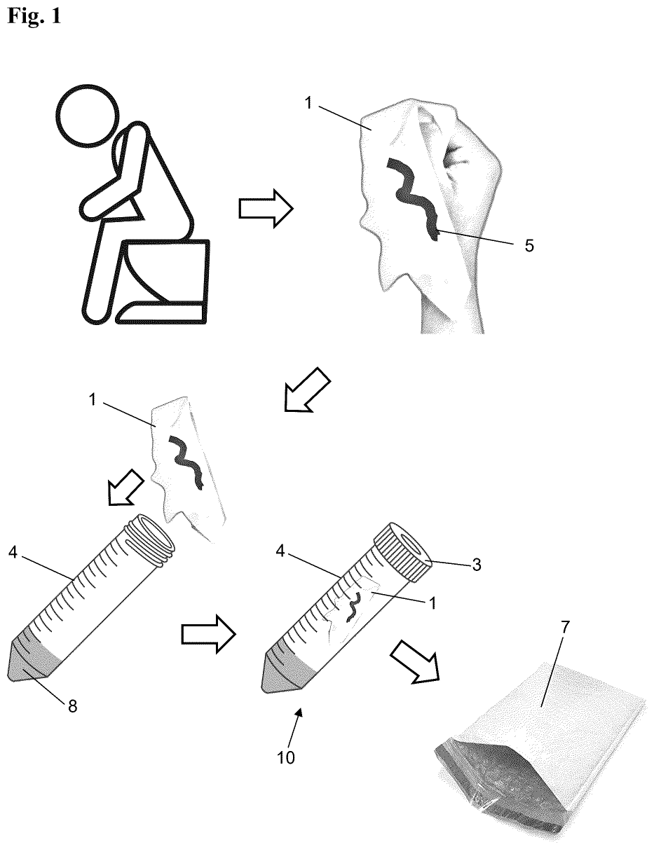

Referring to FIG. 1, an abstraction depicting an overview of a sampling process is provided. In this illustration of a sampling process, a patient defecates and loads a sample 5 of fecal matter onto a swipe 1. The swipe 1 loaded with the sample 5 is then disposed into the body 4 of a sample vial 10. In this instance, the body 4 includes a volume of preservative 8. The body 4 is then sealed by placement of a cap 3 over the open end. The resulting sealed vial 10 is then disposed into a shipping container 7. In this illustration, the shipping container 7 is a mailing envelope.

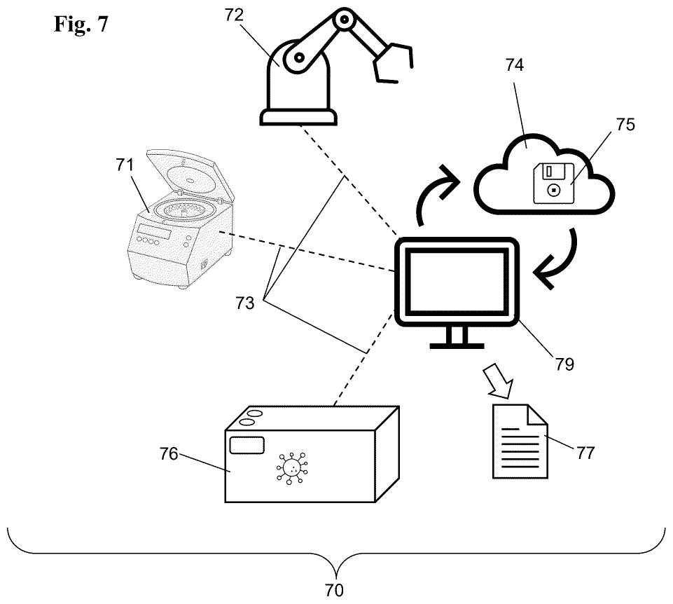

Generally, the swipe 1 and vial 10 (i.e., combination of the body 4, cap 3 and preservative 8) are provided to the patient as a kit (see FIG. 7). In order to enable accurate sample analysis, aspects of the kit may be characterized in advance. For example, by providing a lint-free swipe 1, of a known mass, along with a known mass and/or volume of preservative 8, it is possible to readily assess quantity of the sample 5 and therefore reach conclusions regarding the patient. Examples of conclusions that may be reached include analytical sensitivity needed and determination of concentrations for identified molecular components.

More detail regarding a first embodiment of a vial 10 is provided in FIG. 2.

Prior to discussing different embodiments in detail, it should be recognized that the terms “first”, “second” and the like are provided for ease of introduction and illustration. This terminology is not to be considered limiting. Use of hyphenated reference numbers in the drawings is intended to show that certain features may have common appearance and/or functionality between embodiments. Generally, the latter portion of any hyphenated reference number may be associated with a description for a given embodiment. However, it should be recognized by the reader that at least some of the features of one embodiment may be shared with another embodiment.

In FIG. 2, a first embodiment of a vial 10 is shown. In this example, the first vial 10-1 includes a first body 4-1, a first cap 3-1, and a plunger 12. Disposed within the first body 4-1 is a volume of preservative 8.

In some embodiments, when received by a patient, the body 4-1 is sealed with the cap 3-1. The patient will undertake sampling as described with regard to FIG. 1, unseal the cap 3-1 from the body 4-1, and then dispose the swipe 1 into the body 4-1. Once the swipe 1 has been disposed into the body 4-1, the patient will place the plunger 12 into the body 4-1, essentially on top of the swipe 1. Once the plunger 12 has been placed into the body 4-1, the patient will then seal the body 4-1 with the cap 3-1. At this point, the vial 10-1 contains the swipe 1 with sample 5 loaded there on and is ready for shipping.

In some embodiments, the plunger 12 is not provided to the patient but is used by the laboratory processing the sample 5. When the lab receives the shipping container 7 and sample 5 therein, the lab may open the cap 3-1 and decant a portion of sample fluid while adding the plunger 12 prior to centrifugation. In some instances, the plunger 12 is added, centrifugation is performed, and then decanting of the entire accessible volume is performed.

In this example, the body 4-1 includes a coupling feature 9. The coupling feature 9 provides for robust mechanical engagement of the cap 3-1. In this illustration, the coupling feature 9 includes threads. The threads are designed to engage with opposing threads on an inner surface of the cap 3-1. The cap 3-1 may further include features such as a washer for ceiling with a top surface of the body 4-1.

Other types of coupling features 9 may be used. For example, the coupling feature 9 may include at least one detent/ridge combination to provide for frictional engagement. Half turn or quarter turn spring-loaded designs, such as commonly found in containers for medication may be used. At least one washer and/or sealing ring may be included. In short, the coupling features 9 provide the functionality needed to seal the contents of the vial 10 and prevent leakage to the external environment.

In this embodiment, the body 4-1 includes a set of indicia 6. The indicia 6 may include a variety of information. For example, the indicia 6 may include branding information, identity information (such as an identification number, a barcode, a name, and other alphanumeric or symbolic representations), and may further include measurement related markings such as to indicate a volume within the body 4-1. For example, the measurement related markings may include a scale useful for ascertaining milliliters of preservative 8. Similarly, the cap 3-1 may exhibit indicia 6. For example, in some embodiments, the cap 3-1 exhibits indicia 6 that may be used to match the cap 3-1 with the body 4-1.

Generally, the plunger 12 provides for enhanced centrifugation. That is, this first embodiment 10-1 may be placed in a centrifuge once received at a laboratory for processing. In some other embodiments, the plunger 12 is added to the vial 10-1 at the laboratory. For example, the plunger 12 may be added after removing at least some of the sample fluid. It should be recognized that incorporating the plunger 12 at a later stage provides for greater sensitivity when weighing the vial 10 and therefore derivation of the sample size.

The body 4-1, the plunger 12 and the cap 3-1 may be fabricated from any material deemed appropriate. For example, the body 4-1 may be fabricated from an optical grade synthetic material such as polypropylene (PP) or an acrylic plastic. The body 4-1 may be fabricated from glass. The plunger 12 may be fabricated from any type of material deemed compatible with the preservative 8, the swipe 1 and the sample 5. Generally, the plunger 12 may include at least a few perforations to permit upward flow of preservative 8 during centrifugation of the vial 10-1. In some embodiments, the plunger 12 is provided with a substantial mass such that centrifugation encourages preservative 8 to flow from the swipe 1 during processing.

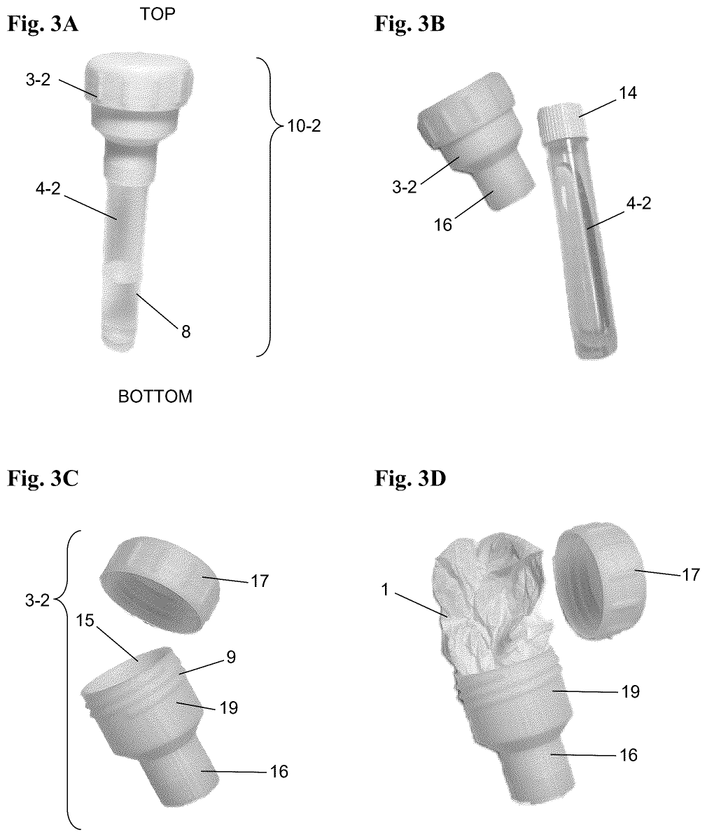

Referring to FIG. 3, a second embodiment of the vial 10-2 is shown. In this example, the body 4-2 exhibits an elongated appearance that is similar to that of a test tube. Shown in the illustration of FIG. 3A, a second embodiment of the cap 3-2 is disposed on top of the body 4-2. As may be seen in the example of FIG. 3B, the cap 3-2 is separable from the body 4-2. The volume of preservative 8 is maintained within the body 4-2 by cover 14.

In this and some other embodiments, the cap 3-2 may be referred to as a “chambered cap” or by other similar terms.

Cover 14 may be provided in various forms. For example, cover 14 may be removed from body 4-2 to make way for installation of cap 3-2. More specifically, and by way of example, cover 14 may be unscrewed from a thread system involving body 4-2. In some embodiments, cover 14 may be designed for engagement with cap 3-2. For example, cover 14 may be designed for frictional engagement for secure engagement with cap 3-2. Features within coupling 16 may provide for piercing cover 14, thus permitting free flow of preservative 8 from body 4-2, through the cover 14 and into a chamber of cap 3-2.

As shown in FIG. 3C, the chambered cap (i.e., cap 3-2) generally includes a storage chamber 15. When lid 17 is removed from shell 19, the storage chamber 15 is revealed. When the storage chamber 15 is exposed, the patient may load the swipe 1 with sample 5 into the interior of cap 3-2 (see FIG. 3D). Once the swipe 1 with the sample 5 has been loaded into the storage chamber 15, lid 17 may be re-engaged with the shell 19 to reassemble the cap 3-2.

Once the cap 3-2 has been reassembled (with the swipe 1 and sample 5 therein), the cap 3-2 is then disposed on body 4-2. Disposition of cap 3-2 onto body 4-2 may proceed by engaging coupling 16 with cover 14 to provide for a liquid tight seal from which the preservative 8 and co-mingled sample 5 may not flow. Accordingly, patient may then invert the vial 10-2 and/or shake vigorously to ensure mixing of the preservative 8 with the sample 5 contained within the cap 3-2.

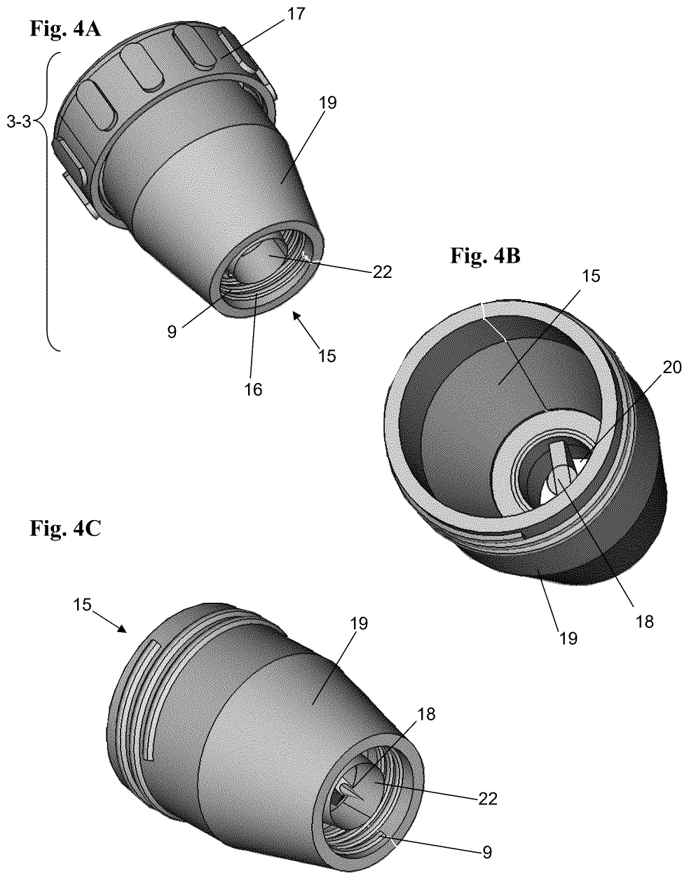

Other features that may be incorporated into embodiments of the cap 3 are depicted in FIG. 4.

In FIG. 4A, aspects of third embodiment of the cap 3-3 are shown. In this example, the cap 3-2 includes lid 17 and shell 19. Included within shell 19 is storage chamber 15. Generally, storage chamber 15 is exposed to coupling 16, which provides for free flow of preservative from the body (see, for example, body 4-2 above) when engaged therewith. Included in this embodiment of the cap 3-3 are coupling features 9. Specifically, in this example, the coupling features 9 include a series of threads. Coupling the cap 3-3 to the body 4-2 simply calls for screwing the cap 3-3 on to the body 4-2 (and may call for first removing the cover 14). Also shown in this example, is a sealing ring 22. Sealing ring 22 may provide a sleeve that penetrates some distance into the body 4-2. Sealing ring 22 may therefore have a length (or height when considered in terms of orientation provided herein) and a taper thus causing tightening of the cap 3-3 over the threads to engage the sealing ring 22 with the inner walls of the body 4-2 and thus providing a liquid tight seal between the cap 3-3 and the body 4-2.

The depiction of the shell 19 in FIG. 4B clearly illustrates thruway 20. Thruway 20 provides for the free flow and co-mingling of preservative 8 with the sample 5 once the components of the vial 10 have been assembled.

Referring also to the depiction of FIG. 4C, it may be seen that the cap 3-3 includes engagement feature 18 for engaging cover 14. In this example, the engagement feature 18 may be referred to as a “bayonet.” As the cap 3-3 is threaded onto the body 4-2, the bayonet will pierce cover 14 disposed onto the body 4-2. One or more engagement features 18 may be included. Included in this embodiment is sealing ring 22. Installing the cap 3-3 with engagement feature 18 will cause breakage of cover 14, which in turn will permit the free-flow of the preservative 8 between within the body 4-2, into cap 3-3.

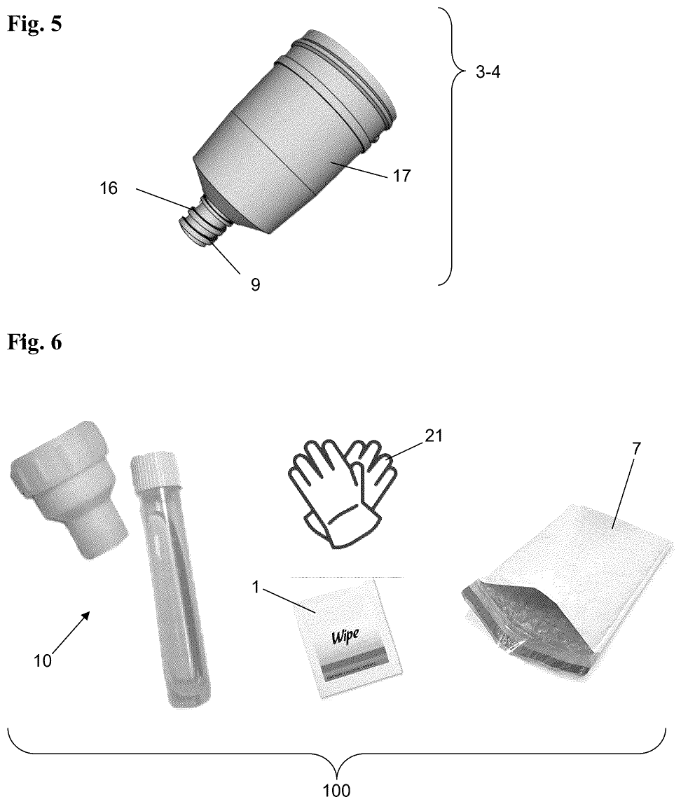

Aspects of yet another embodiment are depicted in FIG. 5. In this example, coupling 16 includes coupling features 9 as threads disposed on an exterior surface thereof. Accordingly, coupling 16 may also be configured to provide the function of the sealing ring 22. That is, coupling 16 may threadably engage with an interior of the body 4-2. Not shown in this example is the lid 17.

As depicted in FIG. 6, in some embodiments, a sampling kit 100 is provided. Generally, the sampling kit 100 includes the cap 3, a sealed body 4 loaded with a known volume of preservative 8, at least one swipe 1, and a return shipping container 7. The kit may include an adequate supply of caps 3, bodies 4 and swipes 1 to provide for collection of multiple samples. Once each sample 5 has been collected, and a sample vial 10 assembled therefrom the vials 10 containing respective samples 5 may be shipped to a laboratory for analysis.

In some embodiments, the sampling kit 100 may include non-transitory machine-readable media with a set of machine-readable instructions contained herein. This set of instructions may be provided as a “kit-chip” (not shown). For example, the kit-chip may include a data set that provides information on contents of the particular sampling kit 100. The kit-chip may include an RFID antenna and thus be enabled to record aspects of shipping history. In some instances, the kit-chip provides downloadable information to a laboratory when received. The downloadable information may include aspects such as history, patient identity, weight and volume data and other such data. In some embodiments, the kit-chip is included in the shipping container 7. In some other embodiments, the kit-chip may be included in a components such as the cap 3. In some instances, the data set is provided in written form used for manual entry, or in another form. For example, the data set pertaining to the kit 100 may be retained in a central database and associated via a bar code or other type of marking.

When supplied with the sampling kit 100, the patient may collect the sample 5 using the supplied swipe 1 and hygienic materials 21, such as gloves. Sample collection may be conducted in private, with the patient then loading the swipe 1 with the sample 5 into one of the body or cap (as appropriate). The patient would then set the cap 3 onto the body 4, thereby wetting the sample 5 with preservative 8. Once the sample 5 has been adequately wetted with preservative 8, such as by shaking of the vial 10 (i.e., the assembly of the cap 3 and the body 4 with the sample therein), the vial 10 is then disposed into a shipping container 7 and shipped to an appropriate laboratory for analyses. For purposes of simplicity and to avoid confusion, an assembly of the cap 3 and the body 4 may be referred to herein as a “sample vial” 10 or simply “vial” 10. Once the cap 3 has been securely disposed onto the body 4, mixing may be performed.

For purposes of discussion herein, the term “sample fluid” and other similar terms refers to a mixture of the sample 5 with the preservative 8 after mixing. Generally, in some embodiments, analysis of the sample 5 calls for analysis of the sample fluid. It may be advantageous to analyze the sample fluid within the vial 10, without further manipulation thereof. In some embodiments, it may be advantageous to decant the sample fluid from the vial 10. More specifically, it may be advantageous to decant supernatant sample fluid and load an appropriate sample carrier, such as a sample bottle configured for particular instrumentation.

Generally, “supernatant” refers to the clear liquid that remains above a solid or precipitate after a mixture has been centrifuged, allowed to settle, or otherwise separated by gravity or other means. “Supernatant” is used herein to describe the liquid phase in procedures such as centrifugation, precipitation, or sedimentation.

Generally, upon assembly, the vial provides a liquid tight environment that is robust and durable during extended periods of shipping and transport. More specifically, and as supported by various trials evaluating sample quality after shipping through common carriers, it has been seen that collected samples shipped via regular mail without cold chain exhibit stability for up to several weeks. This period is adequate to cover routine shipment as well as some shelf-life while awaiting analysis. Generally, and has been found that the techniques disclosed herein are very effective for preserving metabolites of interest that include short-chain fatty acids (SCFAs) and others.

Once received at the laboratory, vial 10 may be disassembled to access fluid there within. In some embodiments, vial 10 is suited for disposing directly into an analytical device such as a mass spectroscopy unit. Refer to FIG. 7.

Generally, the term “laboratory” refers to a point of at least one of processing and analysis of the vial 10 and sample 5 contained therein. This term is not to be considered limiting. It should be recognized that at least some of the aspects involved in processing and analysis may take place at single purpose facilities providing limited functionality.

As shown in FIG. 7, aspects of an exemplary processing system 70 are shown. In this example, processing system 70 includes sample handling equipment for sample preparation. Equipment for sample preparation includes a centrifuge 71 and robotic equipment 72. The robotic equipment 72 may perform a variety of functions. For example, robotic equipment 72 may provide unpacking of shipping containers 7, weighing of each vial 10, and recording weight data (which is used during analysis for data processing). Unpacking shipping containers 7 may call for numerous mechanical manipulations, as well as machine vision functions necessary for recognizing sample identification information, sample volume, and other such aspects. Robotic equipment 72 may further undertake loading of analysis equipment 76. Analysis equipment 76 may include, for example, equipment adapted for: gas chromatography-mass spectrometry (GC-MS) in order to identify and quantify volatile compounds; liquid chromatography-mass spectrometry (LC-MS) in order to identify non-volatile and complex small molecules; nuclear magnetic resonance (NMR) spectroscopy, in order to determines molecular structure and dynamics; and Fourier transform infrared (FTIR) spectroscopy in order to identify functional groups in molecules. Other techniques may be used.

Generally, the processing system 70 includes a central processor 79. The central processor 79 is in communication with components of the processing system 70 through network connections 73. Included is data storage 74, which may include remote data storage equipment. Stored on non-transitory machine readable media within the data storage 74 is a set of instructions which may be referred to herein as an “application” 75. Generally, application 75 includes instructions for implementation by the central processor 79. That is, application 75 may include instructions for any one or more of managing operation of the centrifuge 71 (and any other sample preparation equipment), robotic equipment 72 as needed for machine oriented processing, interfacing with the analysis equipment 76, and generation of a report 77. The central processor 79 may include other components as appropriate such as connection to a user interface. In some embodiments, the application 75 is configured to remotely control central processor 79, in others the application 75 is configured to run on the same hardware. In some embodiments, the application 75 is accessible via a browser and configured for network interfacing. The application may include at least one application program interface (API) for providing network functionality between disparate types of equipment.

Generally, when a sample is received for processing, the central processor 79 will associate the sample with a particular sampling kit 100. For example, the central processor 79 may read the ID number on the vial 10. The central processor 79 will then query a data table stored in the data storage 74 (or in an associated kit-chip). The data table includes, for example, kit data for the particular sample kit 100. The kit data will include, for example, mass, volume, type data and the like regarding the swipe 1 and the preservative 8 contained in that sample kit 100. The central processor 79 will also use intake weight data (obtained when the kit was returned for processing) with kit data to derive additional sample data. Sample data may include mass of the actual sample 5. The sample data and the kit data will then be used to normalize data returned by the analysis equipment 76 and to produce a report 77.

The report 77 may include identification of molecules determined to be present in the sample 5 and the sample fluid. The application 75 may employ statistical tests as appropriate to make determinations. The application may also call up artificial intelligence technologies to analyze data returned by the analysis equipment 76.

The report 77 generally provides metabolite profile information containing at least one of molecular identity information, concentration information. Additionally, the report 77 may suggest pathology information associated with the presence or absence of any particular metabolites or group of metabolites. In some embodiments, the system 100 is adapted for decanting supernatant sample fluid and loading an appropriate sample carrier (not shown) such as a sample bottle matched to particular instrumentation (which may be calibrated for use therewith).

Tests of the efficacy of the sampling apparatus as disclosed herein were conducted. In the tests, samples were taken and shipped through regular mail. Comparison of the mailed samples to lab controls were completed. That is, analysis for a short chain fatty acids (SCFA) showed good outcomes for samples of varying mass, density, and over a period of time as may be expected with shipping, and through actual shipping evolutions.

In the tests, sampling was found to capture the compounds of interest such as short-chain fatty acids (SCFAs), p-cresol and other molecules that are expected to be detectable in stool, such as indole, bile acids, vitamins, amino acids, lipids and others. Validation focused on SCFAs and other established diagnostic markers to rigorously assess performance of the method for molecules of clinical relevance. As the test apparatus disclosed herein enables measuring weight of each sample (i.e., the stool weight), the abundances of metabolites could be normalized. With the use of external calibration, absolute quantities for various molecules could be calculated and used as biomarkers to gain lifestyle or other information. The ratios of metabolites are not dependent on the absolute quantitation, and can also be more linked to specific conditions.

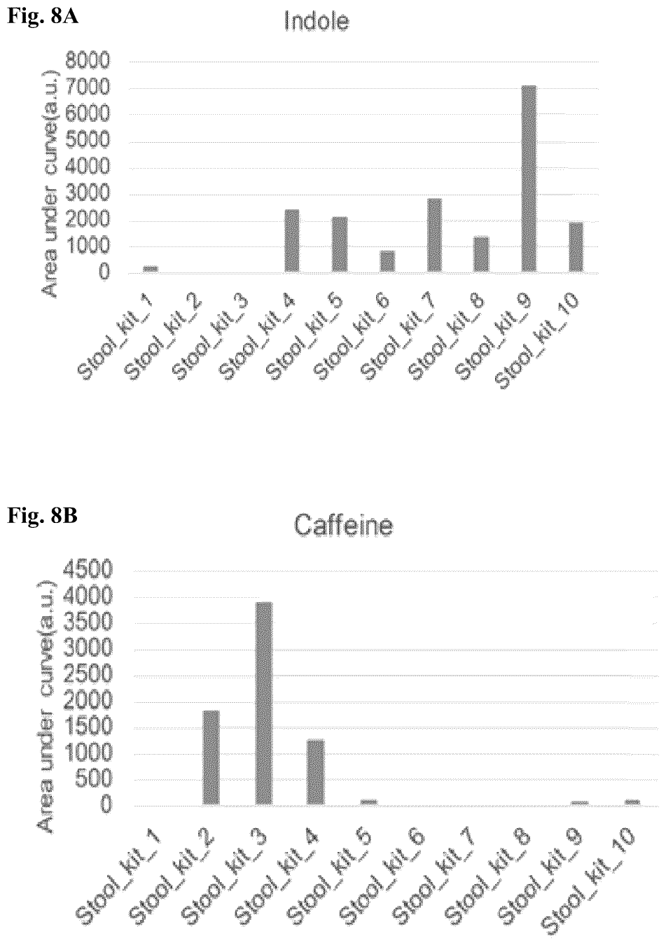

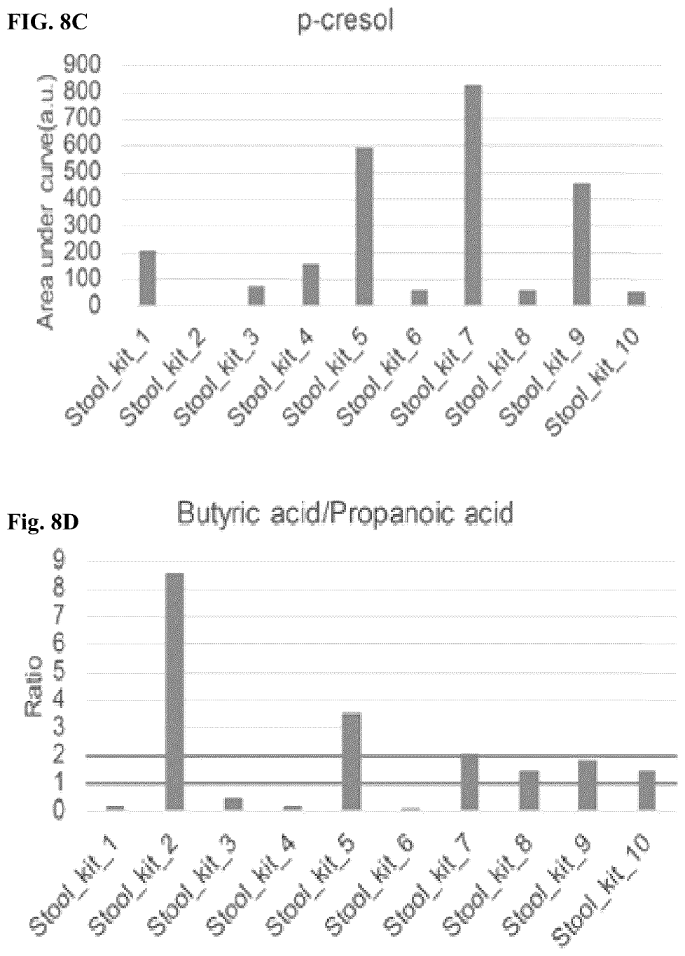

Refer to FIG. 8, which depicts aspects of variability of different molecules captured across test participants. FIG. 8A shows Indole, major smell component of stool. FIG. 8B shows Caffeine, only found in coffee/tea consumers. FIG. 8C shows p-cresol, a breakdown product of protein and proposed biomarker of autism in children. FIG. 8D shows excessively low ratio of butyrate to propionate (subjects below red bar) is associated with insufficient fiber intake, loss of butyrate producing bacteria, inflammatory conditions, while excessively high ratio (subjects above blue bar) may be indicative of small intestinal bacterial overgrowth (SIBO). The expected ratio for a healthy population is 1-2.

All of the studies conducted used were provided as consumer oriented kits and contained lint-free paper (Kimberly-Clark Professional™, 34120), the preservation solution in a 5 ml container spiked with an internal standard, a tube stand, and sampling instruction sheet. The kits were provided to study participants free of charge.

For protocol optimization, all stool samples were provided by healthy volunteer donors. The protocol was run alongside a long sonication and incubation extraction protocol. The long protocol included a ten minute sonication on ice and six hours on a vibration table at two to four degrees Celsius for homogenization. Samples included kits with no wipe, complete kits, and kits with no stool. Samples were generated in triplicates. After sample extraction, samples were analyzed by GC-MS and LC-MS/MS.

For stability optimization, all stool samples were provided by healthy volunteer donors. Samples collected by kits and controls were incubated at twenty five degrees Celsius for 0-, 3-, 5-, and 7-days. Negative controls contained no stool and positive controls contained no stool and Accustandard FAMQ-004 at 1 mM. All samples were collected in triplicate. Sampled kits were stored at minus eighty degrees Celsius until analysis and all samples were extracted together. After sample extraction, samples were analyzed by GC-MS and LC-MS/MS.

For a shipping study, all stool samples were provided by healthy volunteer donors. Samples collected by kits and controls were sent and returned via United States Postal Service to a short-distance (36-miles) and long-distance (2,950-miles) address. Negative control contained no stool. All samples were in triplicates. Upon reception in the mail, the kits were stored at two to four degrees Celsius until analysis. All samples were extracted and analyzed by GC-MS and LC-MS/MS at the same time.

Sample extraction techniques were evaluated as well. Different parameters such as the order of steps, solvent volume, and centrifugation time and setting were tested to establish the standard protocol, which was selected as the simplest and most effective approach. Completed kits were kept at minus eighty degrees Celsius and were thawed on ice prior to extraction. Pre-sample and post-sample kit mass was recorded. The completed kits were first placed in an iced ultrasonic bath for ten minutes and then on a vibration table for ten minutes at room temperature for homogenization. 1000 μL aliquot was taken and transferred to 1.5 mL microcentrifuge tubes. The samples were centrifuged for five minutes at 14,000 revolutions per minute. After centrifugation, aliquots of 100 μL were taken from each sample and transferred to vials with conical inserts, and analyzed by GC-MS and/or LC-MS/MS. Samples were directly loaded onto GC-MS. For LC-MS/MS, samples were diluted four-fold with pure cold methanol to precipitate protein. Samples then were filtered through Phenomenex Phree plates with the application of four psi negative pressure. 200 μL of collected filtrates were dried under vacuum and reconstituted with 100 μL of C18 resuspension buffer (five percent Acetonitrile (Sigma, USA) in LCMS grade water with added internal standards: Sulfachloropyridazine (TCI, USA) and Sulfamethazine (Sigma, USA)).

As to GC-MS data acquisition and processing, 1 μL aliquot of the kit supernatant was directly injected into an Agilent 6890 GC interfaced to a mass spectrometer Hewlett Packard MSD 5973 for electron ionization GC-MS. The GC utilizes a 30 m ZB-FFAP column (0.25 mm i.d., 0.25 μm film thickness) for metabolite separation with 1.2 mL/min constant He flow. The oven temperature program initiates at 50° C. rising to 240° C. at 10° C./min. No noticeable carryover was observed over the entire injection sequence for all of the studies. Also, no increased contamination that necessitated liner change was observed, indicating that possible lint particulate traces from wipe material did not contribute no observable analytical interference. The data were then deconvoluted with the MSHub algorithm. The experimental spectra were searched against the NIST 2023 library with ≥80% spectral match defining putative identifications, and the retention times falling within <0.01 min of the corresponding reference standards. Targeted analysis of SCFAs and p-cresol was performed by using Agilent Masshunter software.

As to LC-MS/MS data acquisition and processing, the samples were injected and chromatographically separated using a Vanquish UPLC (Thermo Fisher Scientific, Waltham, MA), on a 100 mm×2.1 mm Kinetex 1.7 μM, C18, 100 Å chromatography column (Phenomenex, Torrance, CA), 40° C. column temperature, 0.4 mL/min flow rate, mobile phase A 99.9% water (J. T. Baker, LC-MS grade), 0.1% formic acid (Thermo Fisher Scientific, Optima LC/MS), mobile phase B 99.9% acetonitrile (J. T. Baker, LC-MS grade), 0.1% formic acid (Fisher Scientific, Optima LC-MS), with a the following gradient: 0-1 min 5% B, 1-8 min 100% B, 8-10.9 min 100% B, 10.9-11 min 5% B, 11-12 min 5% B.

MS analysis was performed on Orbitrap Exploris 240 (Thermo Fisher Scientific, Waltham, MA) mass spectrometer equipped with HESI-II probe sources. The following probe settings were used for both MS for flow aspiration and ionization: spray voltage of 3500 V, sheath gas (N2) pressure of 35 psi, auxiliary gas pressure (N2) of 10 psi, ion source temperature of 350° C., S-lens RF level of 50 Hz and auxiliary gas heater temperature at 400° C.

Spectra were acquired in positive ion mode over a mass range of 100-1500 m/z. An external calibration with Pierce LTQ Velos ESI positive ion calibration solution (Thermo Fisher Scientific, Waltham, MA) was performed prior to data acquisition with ppm error of less than 1. Data were recorded with data-dependent MS/MS acquisition mode. Full scan at MS1 level was performed with 30K resolution. MS2 scans were performed at 11250 resolution with max IT time of 60 ms in profile mode. MS/MS precursor selection windows were set to m/z 2.0 with m/z 0.5 offset. MS/MS active exclusion parameter was set to 5.0 s.

LC-MS raw data files were converted to mzML format using msConvert (Proteo Wizard). MS1 features were selected for all LC-MS data sets collected using the open-source software MZmine 3 with the following parameters: mass detection noise level was 10,000 counts, chromatograms were built over a 0.01-min minimum time span, with 5,000-count minimum peak height and 5-ppm mass tolerance, features were deisotoped and aligned with 10-ppm tolerance and 0.1-min retention time tolerance, and aligned features were filtered based on a minimum 3-peak presence in samples and based on containing at least 2 isotopes. Subsequent blank filtering, total ion current, and internal standard quality control were performed.

### Results.

Sampling was found to capture the compounds of interest such as SCFAs, p-cresol and other molecules that are expected to be detectable in stool, such as indole, bile acids, vitamins, amino acids, lipids and others. While molecular networking demonstrates that the techniques preserve the broader stool metabolome, validation focused on SCFAs and other established diagnostic markers to rigorously assess performance for molecules of immediate clinical relevance. As the techniques provide for measuring stool weight, the abundances of metabolites can be normalized. Also, with the use of external calibration data, absolute quantities for various molecules could be calculated and used as biomarkers to gain lifestyle or other information. The ratios of metabolites are not dependent on the absolute quantitation, and can also be more closely correlated to specific conditions.

Stability during storage. To evaluate performance for SCFA analysis, the stability of these metabolites were first assessed under room temperature storage conditions. While SCFAs are generally stable at room temperature, their preservation specifically within the sampling matrix had not been validated. Two complementary experiments were conducted. First, sampling matrix was spiked with a 500 μl aliquot of Accustandard FAMQ-004 standard (New Haven, CT USA) at 10 mM concentration to track recovery of known SCFA concentrations. Second, stool samples were collected using sampling materials to assess stability of endogenous SCFAs. Both the standard-spiked wipes and stool-sampled wipes were stored at room temperature for periods ranging from one day to one week.

GC-MS analysis revealed notable stability of all nine SCFAs over the study period. Statistical analysis showed an average relative standard deviation of 23.48% across all time points, which demonstrated good technical reproducibility. Importantly, no noticeable degradation was observed during room temperature storage.

Benchmarking. To benchmark the techniques against existing methods, side-by-side comparisons were conducted with conventional stool collection and commercial sampling approaches. Previous studies comparing preservation methods demonstrated that 95% ethanol provides excellent metabolite preservation compared to FOBT and FIT cards, though high ethanol concentration can complicate sample handling and processing. Building on these findings, comparison was performed using 60% ethanol, with direct collection and OmniGene Gut. The latter is chosen as a current commercially available standard for room-temperature metabolomics sampling. The 60% ethanol concentration was chosen to balance preservation with practical handling considerations (particularly reduced flammability), while maintaining similar metabolite recovery to direct collection methods. Three SCFAs with established diagnostic importance-acetic acid, propanoic acid, and butyric acid-were of particular focus while also monitoring other SCFAs and p-cresol. The comparison revealed that the techniques disclosed herein captured these metabolites with comparable or better sample-to-sample consistency than traditional methods. While measured concentrations aligned well across all three collection methods, the techniques demonstrated lower variability in replicate measurements. One notable exception was acetic acid, which showed high variability across all methods, ostensibly due to its high volatility.

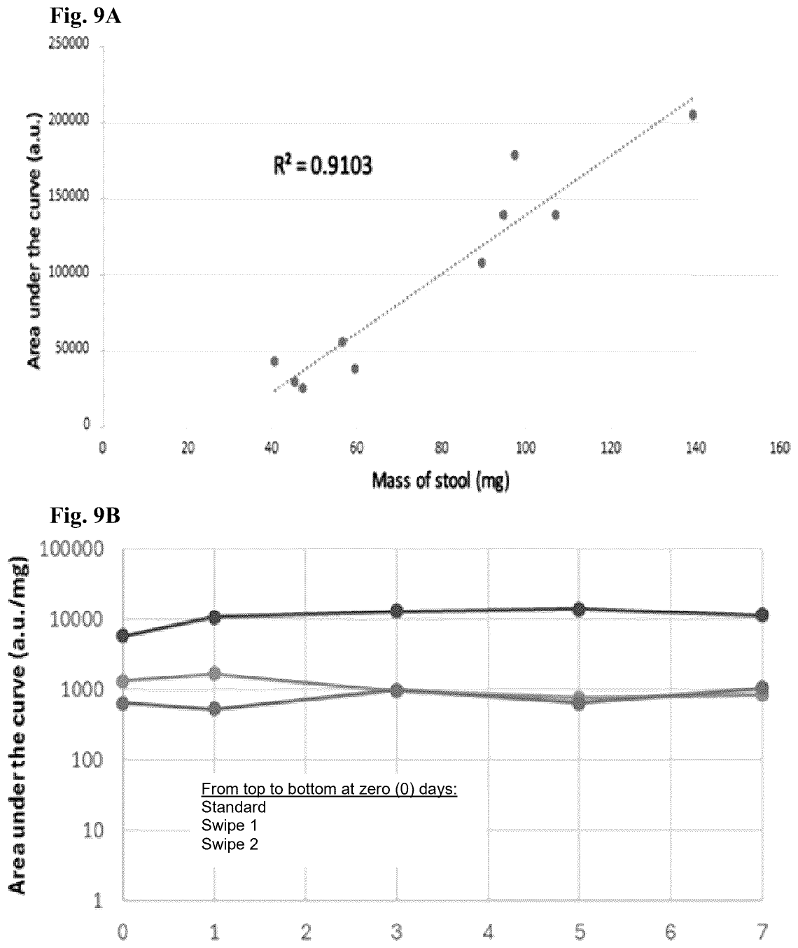

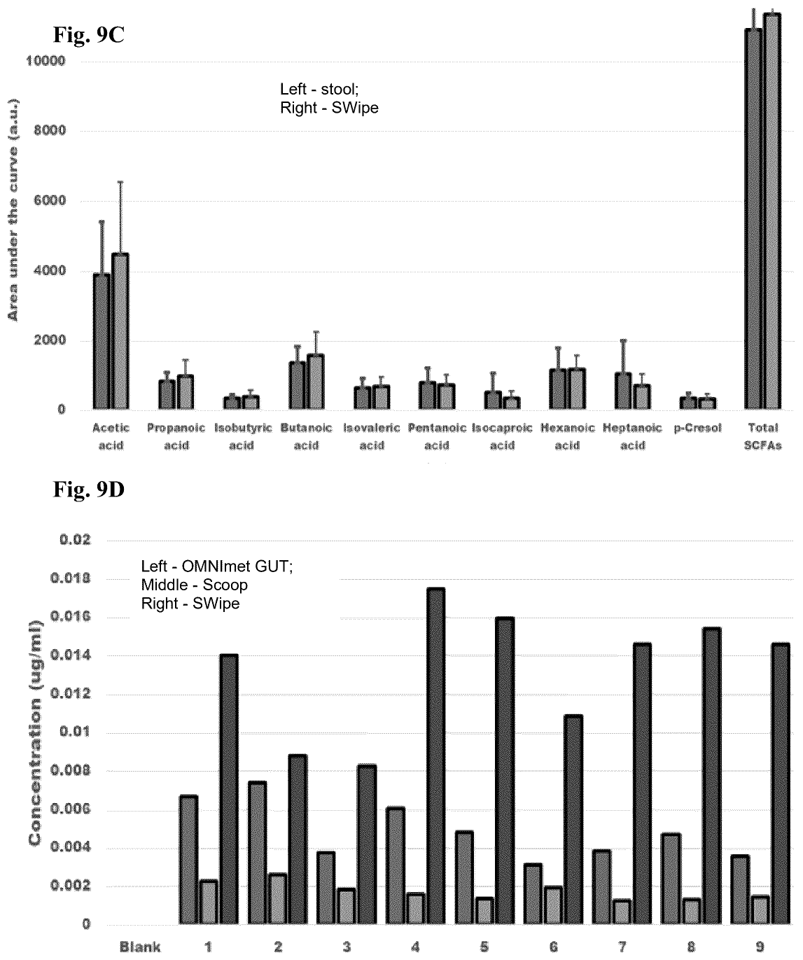

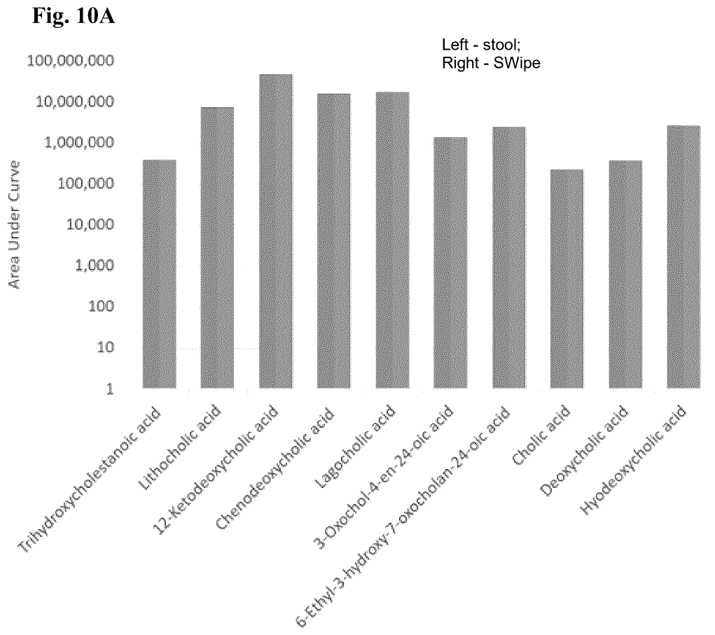

Reference may be had to FIG. 9 which depicts aspects of method comparisons. FIG. 9A depicts a catter plot of total abundance of C₂-C₇ SCFAs versus the measured mass of collected samples for both stool and the kit. A significant positive correlation ((Pearson's R=0.95, p<0.001)) is evident. FIG. 9B depicts stability of SCFAs over time. FIG. 9C depicts a comparison of variability in SCFAs for stool collected with scooping method (conventional sample collection) versus collection techniques disclosed herein. FIG. 9D depicts a plot showing the total concentration of SCFAs in supernatant for different users, normalized by the stool weight.

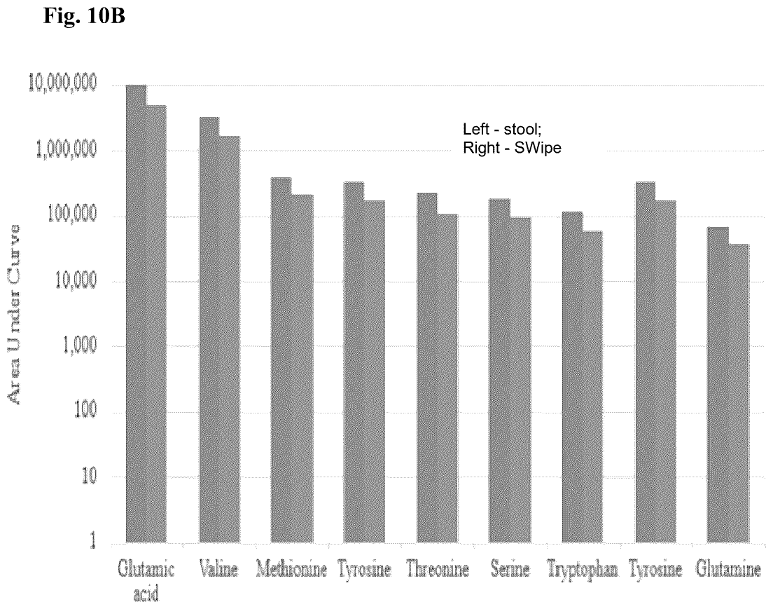

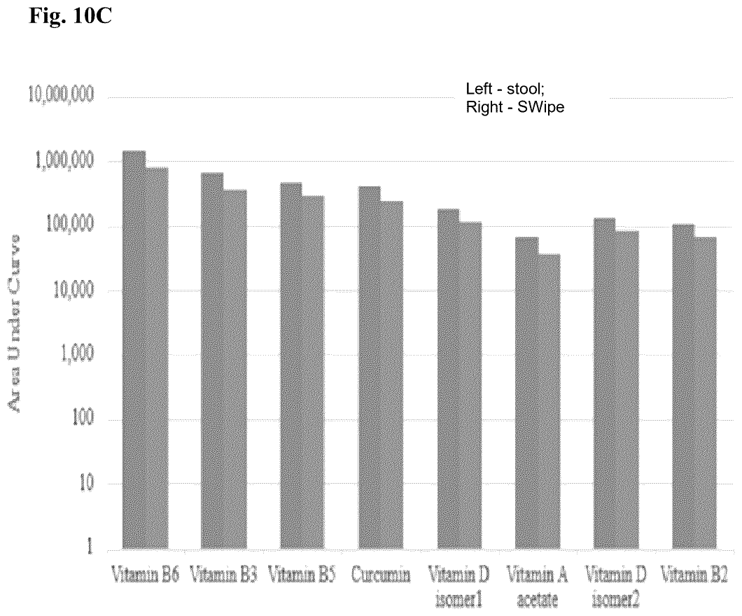

FIG. 10 is a series of graphs comparing data for conventional sampling with techniques disclosed herein. Specifically, LC-MS collected data comparing amounts of various metabolites detected with a various protocols for bile acids, amino acids and vitamins. The abundance of the captured metabolites for both regular direct stool collection and techniques disclosed are similar.

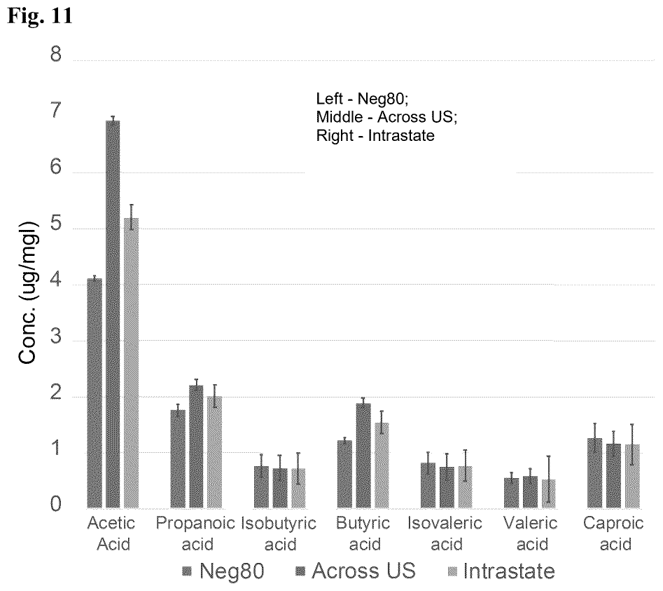

FIG. 11 provides graphic representation of stability data. In FIG. 11, a bar plot showing the distribution of short- and medium-chain fatty acids as well as p-cresol and phenol across three different conditions in the shipping study is provided. “Neg80” correlates to samples stored at minus eighty degrees Celsius immediately after collection. “Intrastate” correlates to samples shipped within the state (approximately one week passed in between shipping and receiving samples). “Across US” correlates to samples shipped from East to West coast of the US (approximately two weeks passed in between shipping and receiving samples). The “Intrastate” and “Across US” samples were stored between about two to four degrees Celsius upon arrival to the lab until analysis. All of the samples were analyzed by GC-MS. Analyses were conducted in triplicate.

These findings indicate that the collection techniques described herein preserve the native stool metabolome without significant alteration. After appropriate normalization and blank subtraction to account for background signals, data from samples should be directly comparable to direct sampling methods that do not bias metabolome composition.

Cell debris removal. To confirm the suitability of the apparatus and method as disclosed herein for streamlined metabolomics workflows, compatibility with mass spectrometry (MS) analysis without the need for extensive sample manipulation and cleanup was assessed. In MS-based metabolomics, various methodologies such as lyophilization/reconstitution, solid-phase extraction, and bi-phase extraction are commonly employed to remove unwanted background components, including proteins, lipids, and cell debris, which can interfere with MS analysis by causing clogging, introducing background noise, and affecting analyte solubility and ionization efficiency.

Fecal samples naturally contain a large number of host and microbial cells that must be removed before introduction into the MS system. Unlike direct fecal sample collection methods, the approach disclosed herein is designed to retain the fecal material within the matrix of the wipe while allowing soluble metabolites to partition into the solvent. It was hypothesized that this feature of the techniques result in a lower cellular content in the collected samples compared to traditional fecal collection techniques. To test this hypothesis, a comparative cell counting analysis was conducted (including both native feces and supernatant) between samples obtained using the approach disclosed herein and those collected through conventional methods.

Supplemental FIG. S9 is showing mass of stool collected in milligrams on the secondary axis (blue bars) as well as Normalized density (ND) of samples as determined by CellsBin proprietary technology (description is given in the Methods section) of real time cell counting (Supplementary video and data). It is evident that the abundance of metabolites is similar between conventional stool collection and S′Wipe, as discussed above, but the cells are generally absent in S′Wipe samples, ostensibly due to their retention on the tissue. Minimizing the presence of cell debris mitigates the risk of instrumentation contamination and circumvents issues such as clogging that may arise from excessive cellular material, enabling simplified extraction protocol, where a single centrifugation step yields a supernatant that is ready for direct MS analysis.

FIG. 8 depicts a plot of group statistics associated twenty samples. The twenty samples were collected according to a standard protocol and shipped using a variety of common commercially available carriers. Analysis showed excellent correlation for the three short-chain fatty acids evaluated. Associated data is provided in Table 1, below.

TABLE 1
Short-chain Fatty Acid - Coefficient of Variation (%)

| | Acetic acid | Butanoic acid | Propanoic acid |
|---|---|---|---|
| Sample-1 | 2.410489 | 12.25822 | 10.95126 |
| Sample-2 | 2.824202 | 6.817882 | 3.038022 |
| Sample-3 | 1.287381 | 2.898456 | 2.50948 |
| Sample-4 | 1.28919 | 3.678 | 3.151236 |
| Sample-5 | 1.682982 | 10.8249 | 15.7263 |
| Sample-6 | 1.455489 | 6.269424 | 1.997689 |
| Sample-7 | 3.795469 | 24.26208 | 8.492203 |
| Sample-8 | 1.3616 | 3.770338 | 12.16466 |
| Sample-9 | 1.213248 | 3.213421 | 2.012929 |
| Sample-10 | 2.089666 | 5.315024 | 3.045444 |
| Sample-11 | 4.736137 | 21.49399 | 15.73764 |
| Sample-12 | 3.19282 | 9.885278 | 7.731907 |
| Sample-13 | 2.76258 | 15.12098 | 16.82502 |
| Sample-14 | 4.321677 | 8.755364 | 5.791193 |
| Sample-15 | 1.508735 | 4.920957 | 2.806436 |
| Sample-16 | 2.433533 | 8.785616 | 4.554395 |
| Sample-17 | 2.273441 | 7.710997 | 4.362358 |
| Sample-18 | 5.65857 | 12.18654 | 15.84043 |
| Sample-19 | 3.469444 | 5.593437 | 12.67307 |
| Sample-20 | 2.140568 | 6.738867 | 3.91761 |

FIG. 9 and FIG. 10 are graphic depictions from shipping trials. In the shipping trials, samples were collected and shipped according to embodiments disclosed. The samples were analyzed following each shipment and mutually compared. Results are depicted.

Having presented embodiments of an apparatus for fecal sampling, some additional aspects and features are now introduced.

Generally, the swipe is provided as a substantially “lint-free” sample media. Examples of lint-free sample media include the following, as well as combinations thereof. Synthetic Fiber Swabs, which may include: nylon-tipped swabs (non-shedding and ideal for collecting fine particles or biological samples); polyester-tipped swabs (generally, durable and lint-free, suitable for use with solvents or cleaning surface); and foam-tipped swabs (which are generally soft and non-abrasive, designed for precision cleaning or sensitive surfaces). Other media may include wipes or pads such as: polyester wipes (woven or non-woven polyester fabrics with high strength and no linting, used in cleanrooms or for contamination sampling); microfiber cloths (including extremely fine synthetic fibers that are lint-free and suitable for particulate collection); and non-woven polypropylene wipes (which are resistant to tearing and solvents, often used in industrial environments). Further examples may include filter paper or specialized pads, such as: glass fiber filters (offering lint-free material with high chemical resistance, used for swipe tests in hazardous environments); cellulose-free filter paper (designed for clean sampling without fiber contamination); and polycarbonate membranes (used for highly sensitive applications such as residue analysis). Additionally, pre-saturated swabs or wipes, which may include: isopropanol-saturated polyester swabs (ideal for cleaning and sampling in electronic or sensitive environments); as well as pre-wetted non-woven wipes (lint-free, pre-treated with solvents for efficient sample collection and decontamination). Silicone or Teflon-coated materials may include: PTFE-Coated Swabs (extremely resistant to chemical interference and lint-free); as well as silicone-coated wipes (which provide a non-shedding, reusable option for sensitive tests).

In some embodiments, the body is adapted from vessels used with analytical instrumentation, thus the body is provided as a “dual purpose” container. For example, a mass spectroscopy unit may be suited for receiving vials of particular dimensions and of a particular optical quality. The body may match the manufacturer's requirements, such that the vial 10 may be admitted directly into analytical processing.

Aspects such as the body 4 and/or cap 3 may be adorned with appropriate labeling. Examples include sample numbers, customer ID, date, preservative type, and other such relevant information. Color coding may be used.

The cap, cover and/or lid may be integrated into the body, removable, breakable (as in the case of embodiments that pierce a top portion of the cover) or provided in any other fashion deemed appropriate.

A variety of engagement features include dimples, detents, ridges, splines, threads, interlocks, snaps and combinations thereof. A layer of pliable material may be included, such as to enhance frictional engagement. The components may be press-fit, frictionally engaged, threaded onto, threaded into, or otherwise engaged with each other.

Any of the components disclosed herein may be fabricated from suitable materials such as: polypropylene (which is known for its chemical resistance and low extractables, polypropylene is commonly used for tubes, vials, and containers); polyethylene (often used in containers and bags, polyethylene offers good chemical resistance and is generally inert); glass (high-quality borosilicate glass is used for vials and containers due to its chemical inertness and durability); silicone rubber (often used in sealing components like gaskets and stoppers, silicone rubber is highly biocompatible and resistant to a wide range of chemicals); PTFE (Polytetrafluoroethylene which is chemically resistant and often used for seals and gaskets in sample storage systems and is used for its chemical resistance and low reactivity); polystyrene (which is often used in labware such as petri dishes and culture plates due to its clarity and ease of sterilization); polycarbonate (which provides clarity and strength, used in some types of containers and laboratory equipment); Nylon (which is used in filters and other components where chemical resistance and mechanical strength are required); Silicone (which is often used in tubing and other flexible components due to its biocompatibility and resistance to sterilization methods); Ethylene Oxide (EtO) Sterilizable materials (these include various polymers and composites that can withstand EtO sterilization, which is often necessary for maintaining sterility in storage). Suitability of materials is to be determined by the designer, manufacturer or other similarly interested party.

Generally, materials selected for the components disclosed herein are selected so as to be inert or substantially transparent to the sample and analysis processes.

In some embodiments, the sampling kit is provided with very precise mass and volume data known in advance. Accordingly, the laboratory receiving the sample assembly may simply calculate sample volume and/or mass of the actual sample in order to reach analytical conclusions.

Accordingly, use of a sample and analysis apparatus is disclosed herein permits efficient laboratory operations by increasing: analytical throughput, decreasing handling, and further limiting exposure to potentially biohazardous materials.

The sampling kit provides for simplified, low-cost at-home stool sampling and metabolomics analysis suitable for large-scale gut microbiome studies and regular consumer health monitoring and is reproducible down to 1 mg for wipe weight. That is, by controlling manufacturing quality, laboratories may readily ascertain accurate sample mass.

Advantageously, the sampling techniques disclosed herein avoid reliance on a cold chain or other specialized or particular accommodations for samples. By use of standardized weights and solvents, sample characteristics may be easily determined. Using the sample apparatuses disclosed herein, laboratories may easily process samples through centrifugation and analysis of the supernatant followed by downstream processing. Downstream processing with the apparatuses compatible with both liquid chromatography mass spectrometry (LC-MS) and gas chromatography mass spectrometry (GC-MS).

Generally, as discussed herein, the term “known” refers to a level of knowledge adequate to perform analytical techniques. For example, in common parlance, it may be understood that a particular volume is 1 or 2 milliliters of a fluid. However, that level of knowledge may not be adequate to perform the types of analyses described herein. More specifically, advance knowledge of a volume in the range of microliters may be required in order to reach desired levels of sensitivity in analyses.

Generally, each swipe 1 is provided with a weight or mass of the swipe 1 known in advance of sampling therewith. Accordingly, the laboratory may subtract the known weight or mass of the swipe 1 and/or preservative 8 in order to calculate the weight or mass of the fecal sample 5. Thus, concentrations of any particular metabolite in the fecal sample may be derived by the laboratory.

In some embodiments, “preservation” of the metabolite profile refers to maintaining sample integrity adequate for analytic purposes after transfer with a common carrier, and for a period of at least five (5) days without the protections afforded by the cold-chain (i.e., refrigeration). In some embodiments, preservation may be for a period of at least ten (10) days, in some embodiments, preservation may be for a period of at least fifteen (15) days, in some embodiments, preservation may be for a period of at least twenty (20) days, in some embodiments, preservation may be for a period of at least twenty-five (25) days, in some embodiments, preservation may be for a period of at least thirty (30) days, in some embodiments, preservation may be for an indefinite period without the protections afforded by the cold-chain (i.e., refrigeration).

In some embodiments, “preservation” of the metabolite profile refers to maintaining sample integrity adequate for analytical detectability of at least 99% the metabolite profile in the original sample after transfer with a common carrier. In some embodiments, preservation refers to maintaining sample integrity adequate for analytical detectability of at least 95%, or 90%, or 85%, or 80%, or 75%, or 70% or 65% or 60% of the metabolite profile in the original sample after transfer with a common carrier. Generally, this is with reference to a number of reliably identifiable chemical and/or biological components of the metabolite profile.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicants thus regard any means which can provide those functionalities as equivalent to those shown herein. No functional language used in claims appended herein is to be construed as invoking 35 U.S.C. § 112 (f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

### LISTING OF REFERENCE NUMBERS

1 Swipe
3 Cap
3-1 Cap
3-2 (Chambered) cap
3-3 (Chambered) cap
3-4 (Chambered) cap
4 Body
4-1 Body (variant)
4-2 Body (variant)
5 Sample
7 Shipping container
8 Preservative
9 Coupling features
10 Vial
10-1 Vial (variant)
10-2 Vial (variant)
14 Cover
15 Storage chamber
16 Coupling
17 Lid
18 Engagement feature
19 Shell
20 Thruway
21 Hygienic materials
22 Sealing ring
100 Sampling Kit
70 Processing system
71 centrifuge
72 robotic equipment
73 network connections
74 data storage
75 application
76 analysis equipment
77 Report
79 central processor

What is claimed is:

1. A vial adapted for preservation of a metabolite profile of a fecal sample during transport thereof, the vial comprising:
a body comprising a volume of preservative; and,
a cap configured to engage with the body and to seal sample fluid comprising a mixture of the preservative and fecal sample within the vial during transport by a common carrier.

2. The vial as in claim 1, configured for receiving a swipe adapted for retention of the fecal sample.

3. The vial as in claim 1, wherein the metabolite profile is associated with at least one of a type of pathogen, a type of bacteria, a type of virus, a gastrointestinal infection, an inflammatory bowel disease, a parasite, a digestive disorder, a gut microbiome, and cancer.

4. The vial as in claim 1, wherein the preservative comprises at least one of water, ethanol, methanol, sodium acetate, formalin, polyvinyl alcohol (PVA), a Cary-Blair medium and sodium thioglycolate.

5. The vial as in claim 1, wherein at least one of the body and the cap comprises at least one of dimples, detents, ridges, splines, threads, a snap, an interlock and at least one sealing feature.

6. The vial as in claim 5, wherein the sealing feature comprises at least one a sealing ring and a separate washer.

7. The vial as in claim 1, wherein the body comprises a cover adapted for retention of the preservative until installation of the cap, thereupon causing a free-flow of the preservative between the cap and the body.

8. The vial as in claim 7, wherein the cap comprises an engagement feature for causing breakage of the cover.

9. The vial as in claim 1, further comprising a plunger configured for compressing a swipe containing the fecal sample upon centrifugation of the vial.

10. The vial as in claim 1, wherein the body is configured for receiving a swipe containing the fecal sample.

11. The vial as in claim 1, wherein the cap comprises a chamber configured for receiving a swipe containing the fecal sample.

12. The vial as in claim 1, wherein the body is configured for evaluation by analytical equipment of a laboratory.

13. A kit for collecting a fecal sample, the kit comprising:
a vial adapted for preservation of a metabolite profile of a fecal sample during transport thereof, the vial comprising: a body comprising a volume of preservative; and, a cap configured to engage with the body and to seal sample fluid comprising a mixture of the preservative and fecal sample within the vial during transport by a common carrier; and,
a swipe for collection of the fecal sample, the swipe configured for being loaded into the vial.

14. The kit as in claim 13, further comprising at least one of hygienic accessories and a shipping container.

15. The kit as in claim 13, wherein the swipe is substantially lint-free.

16. A system for ascertaining metabolite profile information from a fecal sample, the system comprising:
sample handling equipment for analytical preparation of the fecal sample, the fecal sample provided in a vial configured for transport by a common carrier, the vial comprising a body and a cap engaged with the body and sealing sample fluid comprising a mixture of preservative and a swipe with the fecal sample therein;
analysis equipment suited for analyzing the prepared fecal sample; and
a processor comprising machine executable instructions stored on non-transitory machine readable media, the instructions for at least one of operating the sample handling equipment, operating the analysis equipment, interpreting output of the analysis equipment and providing a report containing the metabolite profile information.

17. The system as in claim 16, wherein the analysis equipment is configured for at least one of gas chromatography-mass spectrometry (GC-MS); liquid chromatography-mass spectrometry (LC-MS); nuclear magnetic resonance (NMR) spectroscopy; and fourier transform infrared (FTIR) spectroscopy.

18. The system as in claim 16, wherein the instructions are configured to adjust the metabolite profile information according to a data set comprising information about at least one of the swipe and the sample fluid.

19. The system as in claim 16, wherein the instructions perform the interpreting by implementation of artificial intelligence (AI).

20. The system as in claim 16, wherein the report contains metabolite profile information containing at least one of molecular identity information, concentration information, and pathology information.
